(19) United States
(12) Patent Application Publication
(10) Pub. No.: US 2023/0404940 A1
(43) Pub. Date: Dec. 21, 2023

(54) INTOXICATION-REDUCING SUPPLEMENTAL MIXTURE

(71) Applicant: Megon Dee, Portland, OR (US)

(72) Inventor: Megon Dee, Portland, OR (US)

(21) Appl. No.: 17/845,251

(22) Filed: Jun. 21, 2022

(51) Int. Cl. A61K 31/05; A61K 31/23; A61K 31/015

(52) U.S. Cl. CPC A61K 31/05; A61K 31/015; A61K 31/23

(57) ABSTRACT

An apparatus of the Intoxication-Reducing Supplemental Mixture, a process of forming the Intoxication-Reducing Supplemental Mixture, and a chemical mixture of the Intoxication-Reducing Supplemental Mixture is disclosed. The process of forming the Intoxication-Reducing Supplemental Mixture includes heating an Oil-derived Medium Chain Triglyceride and Cannabidiol Distillate, and mixing the heated components with drops of at least two different terpenoids.

### BACKGROUND

Some people willingly expose themselves or their pets to Cannabis and Cannabis products through various methods to enjoy the effects Cannabis and Cannabis products can provide. Others may be exposed to Cannabis and Cannabis products accidentally and unwillingly, which can cause panic and fear. There is currently no viable solution to exposure to Cannabis and Cannabis products.

### SUMMARY

Disclosed herein are embodiments of an apparatus, process, and chemical mixture to reduce symptoms associated with exposure to Cannabis and Cannabis products. Exposure to Cannabis and Cannabis products could lead to discomfort. The Intoxication-Reducing Supplemental Mixture is used to reduce such symptoms and reduce the time associated with symptoms incurred after exposure to Cannabis and Cannabis products.

The above summary is not intended to describe each illustration embodiment or every implementation of the present disclosure.

### BRIEF DRAWING OF THE DRAWINGS

FIG. 1 illustrates a flow chart demonstrating an example process of creating the chemical mixture for addressing symptoms induced by exposure to Cannabis and Cannabis products, according to inventive principles of this present disclosure.

FIG. 2a illustrates an example chemical equation wherein Cannabidiol may be heated, according to inventive principles of this present disclosure.

FIG. 2b illustrates an example chemical equation wherein an Oil-derived Medium Chain Triglyceride may be heated, according to inventive principles of this present disclosure.

FIG. 3 illustrates an example chemical equation of a heated Cannabidiol and a heated Oil-derived Medium Chain Triglyceride may be combined to create an intermediate chemical mixture, according to inventive principles of this present disclosure.

FIG. 4a illustrates an example chemical structure of Limonene, according to inventive principles of this present disclosure.

FIG. 4b illustrates an example chemical structure of Beta Caryophyllene, according to inventive principles of this present disclosure.

FIG. 5 illustrates an example chemical formula of components being mixed together to form the chemical mixture, according to inventive principles of this present disclosure.

FIG. 6 illustrates an example of the apparatus containing the chemical mixture, according to inventive principles of this present disclosure.

### DETAILED DESCRIPTION

The present disclosure improves the experience of Cannabis and Cannabis product users in multiple ways. Although the present disclosure may describe examples of specific embodiments, one having skill in the art would recognize that the invention should not be limited to those examples.

Cannabis and Cannabis products are currently used medically and recreationally by various users for its various healing and calming effects, but the side effects of Cannabis and Cannabis products may be a problem for some users. Users who are unable to determine the correct dosage or non-users who are accidentally exposed may become fearful and feel unwell due to the exposure to Cannabis and Cannabis products. The effects of Cannabis and Cannabis products could last for hours and traumatize users who may feel like there is no solution to this issue.

An Intoxication-Reducing Supplemental Mixture capable of reducing the effects of Cannabis and Cannabis products may help in reducing discomfort and/or fear associated with usage of, or exposure to, Cannabis and Cannabis products. The Intoxication-Reducing Supplemental Mixture may help bring relief to users by knowing a solution exists in case there is a situation in which they have accidentally been exposed or have taken too large of a dosage of Cannabis or Cannabis products. The Intoxication-Reducing Supplemental Mixture may help in reducing a debilitating or otherwise unpleasant experience by reducing the time the user may feel the effects of Tetrahydrocannabinol exposure. The Intoxication-Reducing Supplemental Mixture is not limited to helping provide solutions to humans but also to other mammalian subjects. The Intoxication-Reducing Supplemental Mixture will help create a more comfortable environment for non-users and users who seek a remedy to any discomfort that may arise when exposed to Cannabis and Cannabis products.

This disclosure describes an apparatus, process, and chemical mixture of the Intoxication-Reducing Supplemental Mixture to be used after being exposed to Cannabis and Cannabis products. The Mixture may reduce symptoms induced by exposure to Cannabis and Cannabis products and reduce the time of discomfort associated with any exposure. The Intoxication-Reducing Supplemental Mixture may reduce any discomfort or fear that may arise when exposed to Cannabis and Cannabis products by reducing the time the user may feel the effects of Cannabis and Cannabis products exposure.

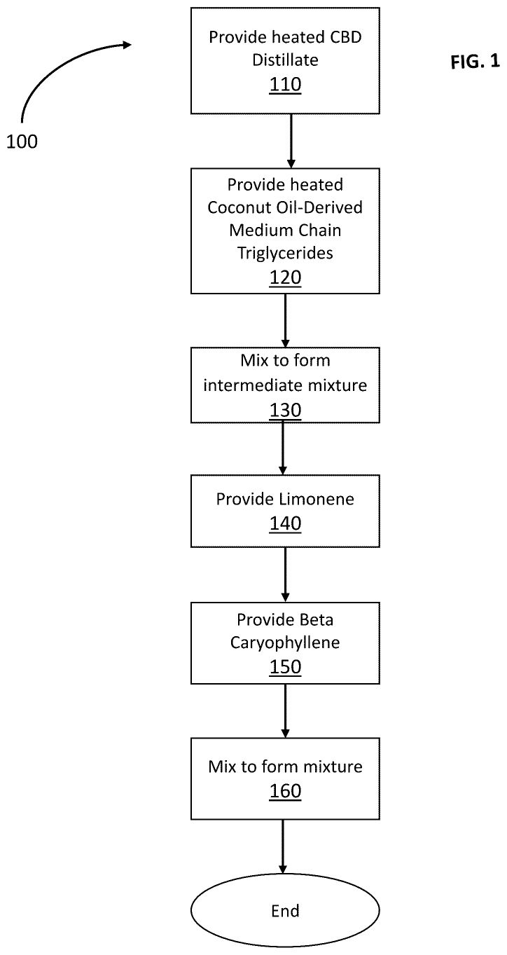

Turning now to FIG. 1, illustrated is a flow chart of an example process 100 for creating the chemical mixture, according to embodiments.

The process begins at 110 with heated Cannabidiol Distillate from hemp, also known as CBD Distillate, being added into a container. A container may include, for example, a glass beaker, a mixing bowl, a flask, a vial, etc. At 120, heated Oil-derived Medium Chain Triglyceride oil, with a preferred embodiment of Coconut Medium Chain Triglyceride Oil also known as Coconut MCT Oil, may be added into the container containing CBD Distillate. Other embodiments may include, but are not limited to, palm kernel oil, coconut butter, coconut milk, and milk, yogurt, butter, cheese, etc.

At 130, the heated CBD Distillate and heated Coconut MCT Oil may be combined in the container using mixing methods, such as a stirring with a vortex mixer, magnetic stirring bar or stirring stick, swirling, shaking, blending, beating, etc. The mixing methods will combine the contents of the container to form an intermediate chemical mixture. The intermediate chemical mixture may then be cooled prior to any additional components being added.

At 140, Limonene may be added to the chemical mixture in the container. Limonene is a terpenoid which may be extracted from citrus fruit peels by cold pressing or distillation methods, such as exposure to steam or boiling water, cooling the resulting vapor, and collecting the condensate. Citrus fruits may include, but are not limited to: oranges, mandarin oranges, tangerines, lemons, grapefruit, clementines, blood oranges, limes, etc. At 150, Beta Caryophyllene may be added to the chemical mixture in the container. Beta Caryophyllene is another terpenoid which may be extracted using gas chromatography from various plants, including, but not limited to: black pepper, oregano, cinnamon, basil, cloves, hops, etc. At 160, all the added components may be mixed at a temperature of 60 degrees Fahrenheit to 80 degrees Fahrenheit at a speed of 150 revolutions per minute, also known as rpm, to 1000 rpm and at an atmospheric pressure of 0 to 2 standard atmosphere, also known as atm, for ten to thirty minutes, with the preferred embodiment of 15 minutes, to form the Intoxication-Reducing Supplemental Mixture. In some embodiments, heat may, or may not, be added during mixing. The preferred embodiment for speed includes 250 rpm to 500 rpm. Mixing may include stirring using a magnetic stirring bar or stirring stick, swirling, using a machine to mix, etc. Once the mixing is completed, the Intoxication-Reducing Supplemental Mixture may be created for reducing symptoms associated with exposure to Cannabis and Cannabis products. The Intoxication-Reducing Supplemental Mixture may act, after proper application to a subject, as a vehicle for reducing symptoms induced by exposure to Tetrahydrocannabinol. In some embodiments, the Intoxication-Reducing Supplemental Mixture may, for example, act as a set of competitive antagonists in relation to psychoactive compounds found in Cannabis and Cannabis products.

The Intoxication-Reducing Supplemental Mixture may be stored in a container, including but not limited to: a spray bottle, glass bottle, plastic bottle, metal bottles, etc. The Intoxication-Reducing Supplemental Mixture is not limited to embodiments in a container, and other example embodiments include, but are not limited to: pills, tinctures, injections, aerosols, nasal sprays, dermal patches, etc. The Intoxication-Reducing Supplemental Mixture may be delivered to the user in various ways including, but not limited to: swallowing, inhalation, absorption through the skin, intravenous injection, etc.

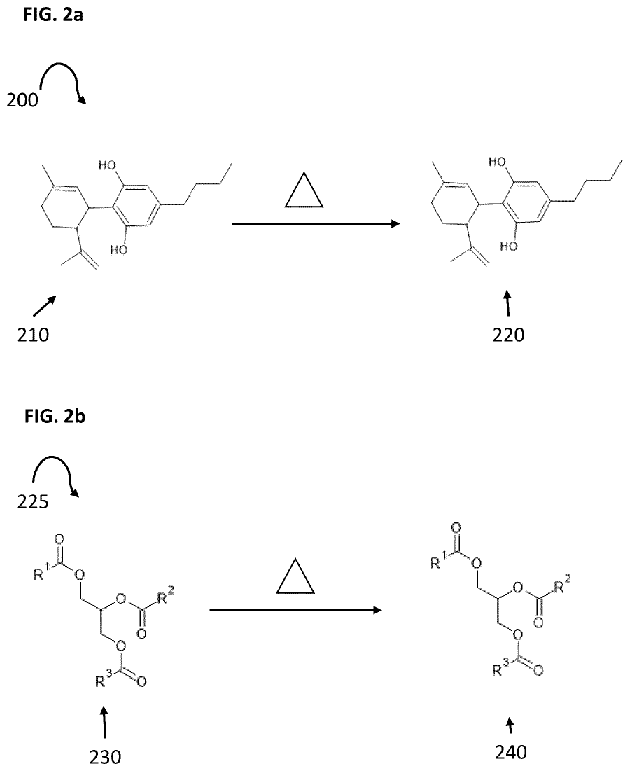

Turning now to FIG. 2a, depicted is an example equation diagram 200 of Hemp-derived CBD Distillate 210 being heated, according to embodiments. In FIG. 2a, Cannabidiol 210 may be heated at a temperature of 100°-300° F. to create a heated Cannabidiol 220 for combining purposes.

Turning now to FIG. 2b, depicted is an example equation diagram 225 of Coconut MCT Oil 230 being heated according to embodiments. In FIG. 2b, Coconut MCT Oil 230 may be heated at a temperature of 100°-300° F. to create a heated Coconut MCT Oil 240 for combining purposes. Coconut MCT Oil is the preferred embodiment for use as an Oil-derived Medium Chain Triglyceride for the intermediate chemical mixture. Heating the different components prior to being combined may help speed up the blending.

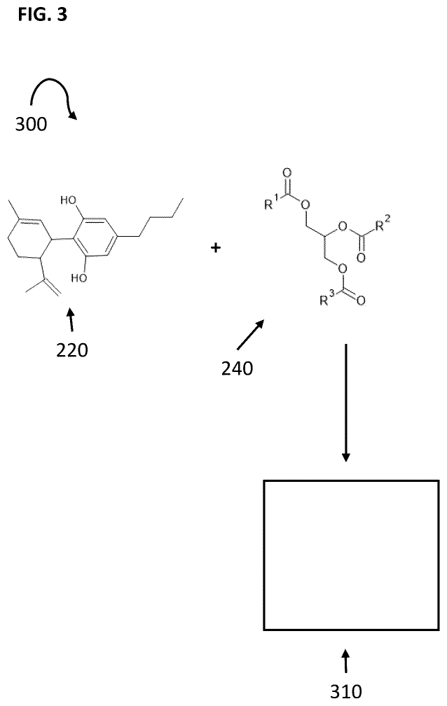

Turning now to FIG. 3, depicted is an example equation diagram 300 showing the combining of heated Cannabidiol 220 and heated Coconut MCT Oil 240, according to embodiments. Combining the heated Cannabidiol 220 and heated Coconut MCT Oil 240 through mixing may create an intermediate chemical mixture 310. This intermediate chemical mixture 310 may then be used to continue the process of making the Intoxication-Reducing Supplemental Mixture.

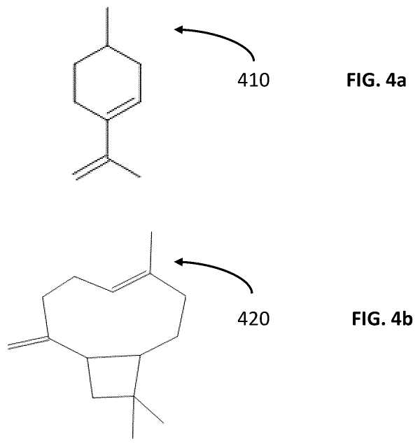

Turning now to FIG. 4a, depicted is an example of a terpenoid with a preferred chemical structure of Limonene 410.

Turning now to FIG. 4b, depicted is an example of a terpenoid with a preferred chemical structure of Beta Caryophyllene 420.

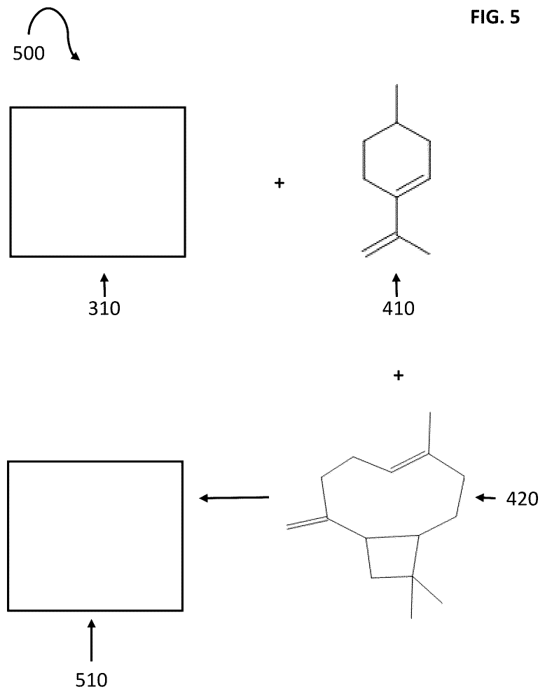

Turning now to FIG. 5, depicted is an example equation diagram 500 showing the combining of the various components of the mixture (intermediate chemical mixture 310, Limonene 410, and Beta Caryophyllene 420) to create the Intoxication-Reducing Supplemental Mixture 510. This Intoxication-Reducing Supplemental Mixture 510 may then be used as a chemical mixture to reduce undesired symptoms associated with exposure to Cannabis and Cannabis products, such as nausea, headache, sleepiness, fear, dizziness, confusion, paranoia, hallucinations, and poor coordination.

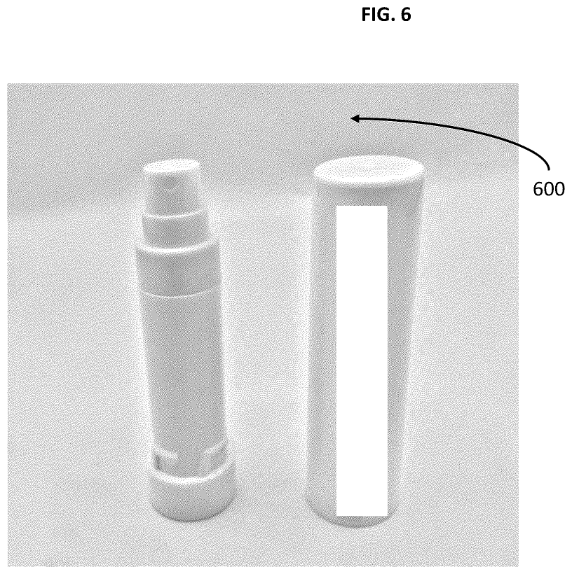

Turning now to FIG. 6, depicted is an embodiment of an example container 600 containing the Intoxication-Reducing Supplemental Mixture. The container containing the Intoxication-Reducing Supplemental Mixture is not limited to spray bottles and may include other containers, such as glass jars, plastic jars, paper bottles, etc.

The flowchart and diagrams in the Figures illustrate the architecture, functionality, and operation of possible implementations of the Intoxication-Reducing Supplemental Mixture according to various embodiments of the present disclosure.

The descriptions of the various embodiments of the present disclosure are presented for purposes of illustration but are not intended to be exhaustive or limited to the embodiments disclosed. A variety of synthetic reactions may be used that vary in reaction components and methods which can ultimately lead to the final formation of Intoxication-Reducing Supplemental Mixture. In some embodiments, processes can be added or changed while still remaining within the scope of the disclosure.

1. An apparatus for reducing symptoms induced by exposure to Tetrahydrocannabinol comprising:
a spray bottle; and
a mixture of a set of components, the set including Coconut Oil-Derived Medium Chain Triglycerides, Cannabidiol, and at least one terpenoid.

2. The apparatus of claim 1, wherein the at least one terpenoid is derived from citrus fruit peels.

3. (canceled)

4. The apparatus of claim 1, wherein the at least one terpenoid includes Beta Caryophyllene.

5. The apparatus of claim 1, wherein said mixture is capable of sublingual administration, via the spray bottle.

6. The apparatus of claim 5, wherein said mixture is administered after exposure to Tetrahydrocannabinol through consumption.

7. The apparatus of claim 5 wherein said mixture is administered after exposure to Tetrahydrocannabinol through smoking.

8. The apparatus of claim 5, wherein said mixture is administered after exposure to Tetrahydrocannabinol by second-hand smoke.

9-13. (canceled)

14. A chemical mixture comprising:
a Cannabidiol;
at least one terpenoid; and
a triglyceride.

15. The chemical mixture of claim 14, wherein the at least one terpenoid is extracted from citrus fruit peels by cold pressing or distillation.

16. (canceled)

17. The chemical mixture of claim 14, wherein the at least one terpenoid is extracted using gas chromatography.

18. The chemical mixture of claim 14, wherein the at least one terpenoid includes Beta Caryophyllene.

19. The chemical mixture of claim 14, wherein the at least one triglyceride is derived from coconut oil.

20. The chemical mixture of claim 14, wherein:
the Cannabidiol is extracted from hemp;
the at least one terpenoid includes:
Limonene extracted from one or more citrus fruit peels from one or more citrus fruits; and
Beta Caryophyllene extracted from one or more plants; and
the triglyceride is obtained from any combination of palm kernel oil, coconut butter, coconut milk, coconut oil, milk, yogurt, butter, and/or cheese.
